(19) United States
(12) Patent Application Publication
(10) Pub. No.: US 2011/0083672 A1
(43) Pub. Date: Apr. 14, 2011

(54) TRACHEOSTOMY TUBE HAVING A CUFFED INNER CANNULA

(75) Inventors: Robert J. Webster, Nashville, TN (US); Todd Dutton, Mt. Airy, MD (US); Sanjay M. Athavale, Nashville, TN (US)

(73) Assignee: Vanderbilt University, Nashville, TN (US)

(21) Appl. No.: 12/855,470

(22) Filed: Aug. 12, 2010

Related U.S. Application Data
(60) Provisional application No. 61/233,280, filed on Aug. 12, 2009.

(51) Int. Cl. A61M 16/04

(52) U.S. Cl. 128/207.15

(57) ABSTRACT

A tracheostomy tube for use with a neck plate having an aperture. The tracheostomy tube comprises an elongate outer cannula having a lumen and configured to extend through the aperture; an elongate inner cannula having a lumen and an inflatable cuff, and configured to extend through the lumen of the outer cannula such that the cuff extends beyond a distal end of the outer cannula; and an interlocking mechanism configured to releasably secure a proximal end of the inner cannula to a proximal end of the outer cannula.

### CROSS-REFERENCE TO RELATED APPLICATION

The present application claims the benefit of U.S. Provisional Patent Application No. 61/233,280 filed Aug. 12, 2009, which is hereby incorporated by reference herein.

### BACKGROUND

1. Field of the Invention

The present invention relates generally to tracheostomy tubes, and more particularly, to a tracheostomy tube having a cuffed inner cannula.

2. Related Art

Tracheostomy tubes are used to provide a direct airway to a patient's trachea when natural respiration is no longer possible. Tracheostomy tubes generally include an elongate cannula, catheter or other tube (generally and collectively referred to herein as “cannula”) formed so as to have a gradual curve to facilitate insertion into a patient's trachea. Typically, an incision commonly referred to as a stoma is made in the front of the patient's neck and trachea, below the patient's larynx and the cannula is inserted through the stoma. When it is located in its operative position, the cannula may be used to provide adequate airway ventilation to the patient. The inserted cannula generally serves one of two purposes: to permit the patient to breathe unassisted; that is, to provide passive ventilation; or to provide an unobstructed passageway so that the patient may breathe with the assistance of a medical device such as a ventilator; that is, to facilitate mechanically-assisted ventilation.

For the ventilation to be effective, it is necessary to force the air into the lungs by preventing the air from escaping into the patient's nasal cavity or mouth. In order seal the airway around the tube, conventional tracheostomy tubes have an inflatable cuff attached to the perimeter of the outer cannula. When inflated, the cuff seals the airway around the cannula, thereby requiring air to pass through only the cannula.

When the tracheostomy tube is properly positioned, the distal end of the cannula extends from the stoma into the patient's trachea. In order to retain the distal end of the cannula in its operative position, the tracheostomy tube generally includes a neck plate or other fixation device which is attached to the proximal end of the cannula. The neck plate typically rests against the exterior surface of the patient's neck and is secured by a collar band extending about the patient's neck.

There are many potential drawbacks with tracheostomy tubes which can range in effect from minor irritation to death. Varying sizes of individuals and differences in anatomical dimensions leads to discomfort from tracheostomy tubes that fit poorly. The rigid ends of the outer cannula often put pressure on the walls of the trachea, which can cause irritation, as well as ulceration. When inflated, the friction between the cuff and the trachea may lead to necrosis of tracheal tissue, as well as to tracheal stenosis, which is a narrowing of the trachea. The detrimental effects of the cuff are unavoidable, and require cuffs to be deflated regularly. Alternatively, cuffed tracheostomy tubes are required to be replaced with uncuffed tubes as soon as possible after the tracheostomy. The process of switching tracheostomy tubes may also be very hazardous, especially when treating obese patients. The stoma can easily be lost among layers of fat and skin, as well as the trachea, which all move independently of one another. Losing the stoma may cause suffocation and possibly death.

### SUMMARY

In accordance with one aspect of the present invention, a tracheostomy tube for use with a neck plate having an aperture is provided. The tracheostomy tube comprises: an elongate outer cannula having a lumen and configured to extend through the aperture; an elongate inner cannula having a lumen and an inflatable cuff, and configured to extend through the lumen of the outer cannula such that the cuff extends beyond a distal end of the outer cannula; and an interlocking mechanism configured to releasably secure a proximal end of the inner cannula to a proximal end of the outer cannula.

In accordance with another aspect of the present invention, a tracheostomy tube is provided. The tracheostomy tube comprises: a neck plate including an aperture; an outer cannula extending through the aperture; a first connector releasably coupled to a proximal end of the outer cannula; an inner cannula extending through the outer cannula and the aperture; a second connector releasably coupling a proximal end of the inner cannula to the outer cannula, wherein each of the cannulae are independently removable from the aperture when the cannulae are in place within a trachea; and an inflatable cuff affixed to a distal end of the inner cannula.

### BRIEF DESCRIPTION OF THE DRAWINGS

Embodiments of the present invention are described below with reference to the accompanying drawings, in which like reference numerals refer to the same or similar components, and in which:

FIG. 1 is a simplified sagittal section of a patient illustrating an exemplary embodiment of a tracheostomy tube of the present invention inserted into the trachea of a patient;

FIG. 2 is a perspective view of the tracheostomy tube illustrated in FIG. 1, in accordance with embodiments of the present invention;

FIG. 3 is an exploded view of the tracheostomy tube illustrated in FIGS. 1 and 2, in accordance with embodiments of the present invention;

FIG. 4A is a perspective view of the inner cannula of the tracheostomy tube illustrated in FIGS. 1, 2 and 3, in accordance with embodiments of the present invention;

FIG. 4B is a perspective view of the outer cannula of the tracheostomy tube illustrated in FIGS. 1, 2 and 3, in accordance with embodiments of the present invention;

FIG. 5A is a perspective view of the outer cannula cap of the interlocking mechanism illustrated in FIGS. 1, 2 and 3, in accordance with embodiments of the present invention;

FIG. 5B is a perspective view of the inner cannula cap of the interlocking mechanism illustrated in FIGS. 1, 2 and 3, in accordance with embodiments of the present invention;

FIG. 6 is a cross-sectional view of the interlocking mechanism illustrated in FIGS. 1-5B, in accordance with embodiments of the present invention;

FIG. 7A is a perspective view of an inner cannula of a tracheostomy tube in accordance with alternative embodiments of the present invention;

FIG. 7B is a perspective view of an outer cannula of a tracheostomy tube in accordance with alternative embodiments of the present invention;

FIG. 8A is a perspective view of an inner cannula of a tracheostomy tube in accordance with alternative embodiments of the present invention;

FIG. 8B is a perspective view of an outer cannula of a tracheostomy tube in accordance with alternative embodiments of the present invention;

FIG. 9A is perspective view of an outer cannula removal tool in accordance with embodiments of the present invention;

FIG. 9B is a close-up view of the outer cannula removal tool of FIG. 9A coupled to an inner cannula, in accordance with embodiments of the present invention;

FIG. 10 is a partial cross-sectional side view of a removal tool and inner cannula, in accordance with embodiments of the present invention;

FIG. 11 is a partial cross-sectional side view of a removal tool and inner cannula, in accordance with embodiments of the present invention;

FIG. 12 is a side view of a removal tool and inner cannula, in accordance with embodiments of the present invention;

FIG. 13A is a flowchart illustrating the steps performed to use a tracheostomy tube in accordance with embodiments of the present invention;

FIG. 13B is a flowchart illustrating two steps of FIG. 13A in greater detail; and

FIG. 13C is a flowchart illustrating two additional steps of FIG. 13A in greater detail.

### DETAILED DESCRIPTION

Embodiments of the present invention are generally directed to a tracheostomy tube usable with a neck plate having an aperture therein. The tracheostomy tube comprises an elongate outer cannula that is configured to extend through the neck plate aperture. An elongate inner cannula having an inflatable cuff is configured to extend through the lumen of the outer cannula such that the cuff extends beyond a distal end of the outer cannula. The tracheostomy tube further includes an interlocking mechanism configured to releasably secure a proximal end of the inner cannula to a proximal end of the outer cannula.

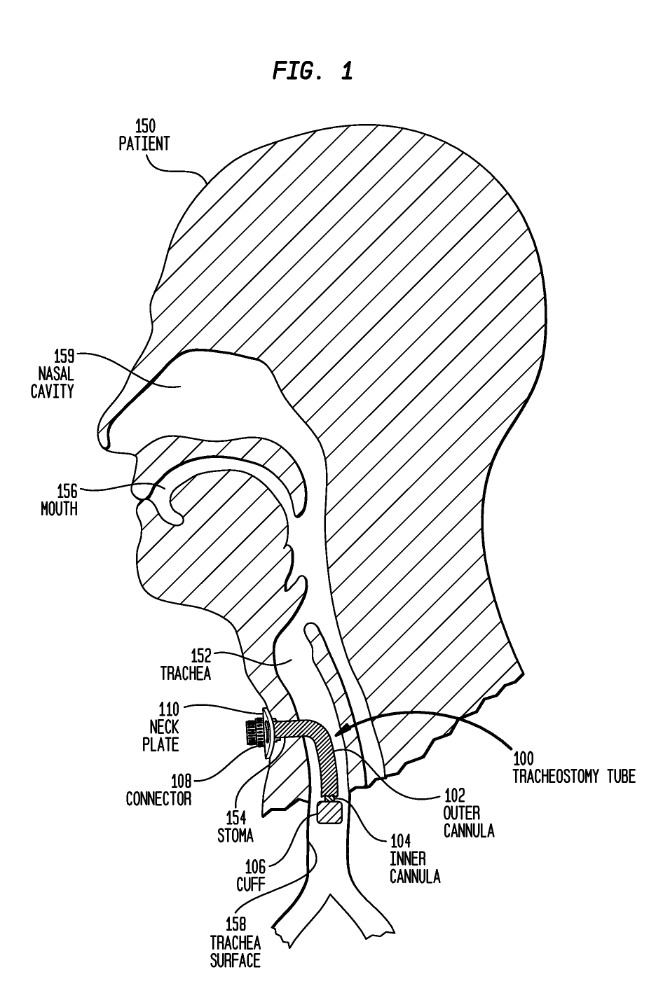

FIG. 1 is a cross-sectional view of a patient 150 illustrating an embodiment of a tracheostomy tube of the present invention, referred to herein as tracheostomy tube 100, inserted into trachea 152 of the patient. Tracheostomy tube 100 is used to maintain an airway when natural respiration is no longer possible. In one instance of use, tracheostomy tube 100 is required when positive pressure ventilation is to be used on patient 150 in situations where orotracheal intubation is not viable.

Tracheostomy tube 100 is a curved tube which is inserted through an incision or stoma 154 in trachea 152. Tracheostomy tube 100 includes an elongate outer cannula 102 and an elongate inner cannula 104. Inner cannula 104 is disposed in and extends through a lumen of outer cannula 102. Both cannulae 102, 104 are inserted through an aperture (not shown) in a neck plate 110. Neck plate 110 provides exterior support to cannulae 102, 104. As is known to those skilled in the art, neck plate 110 prevents cannulae 102, 104 from inadvertently sliding too far into trachea 152. Neck plate 100 may be secured to patient 150 via a strap around the patient's neck or by any other appropriate fastener. The proximal ends of the two cannulae 102, 104 are releasably secured to each other by an interlocking mechanism 108.

For the ventilation to be effective, it is necessary to prevent air from escaping into nasal cavity 159 or out through mouth 156. A cuff 106 is affixed to a distal end of inner cannula 104. The relative lengths of inner cannula 104 and outer cannula 102 is such that cuff 106 extends from the distal end of outer cannula 102. Cuff 105 is an inflatable component. When inflated, cuff 106 expands until it comes into contact with an interior surface 158 of trachea 152. While inflated in such a manner, cuff 106 serves as a barrier, effectively sealing off trachea 152 and preventing air from passing through trachea 152 except through inner cannula 104.

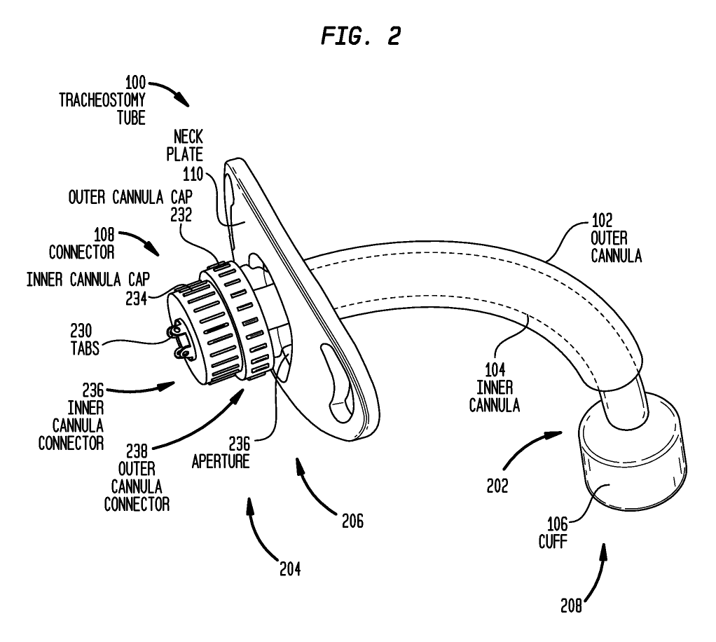

FIG. 2 is a perspective view of tracheostomy tube 100 shown assembled and not inserted in a patient. Both outer cannula 102 and inner cannula 104 extend through an aperture 236 in neck plate 110. Inner cannula 102 is disposed within outer cannula 104 and has cuff 106 affixed to distal end 202 thereof. The proximal ends 204, 206 of inner cannula 104 and outer cannula 102, respectively, are detachably connected to each other using interlocking mechanism 108. In this exemplary embodiment, interlocking mechanism 108 has two parts, an outer cannula connector 238 and an inner cannula connector 236. Each cannula connector 236, 238 comprise a cannula cap 232, 234, respectively, and a cannula collar (not shown). Connectors 236, 238 interlock to prevent relative movement of inner cannula 104 and outer cannula 102. Connectors 236, 238 of interlocking mechanism 108 may be disconnected or detached from each other to enable the cannulae to be repositioned separately removed from the aperture while the other cannula remains in place. Interlocking mechanism 108 is sized larger than the aperture 236 so that when connected to cannulae 102, 104, the cannulae cannot pass through the aperture in neck plate 110, thus helping to ensure the safety of patient 150. Proximal end 204 of inner cannula 104 also includes tabs 230 for attaching a removal tool (not shown) as is described further below.

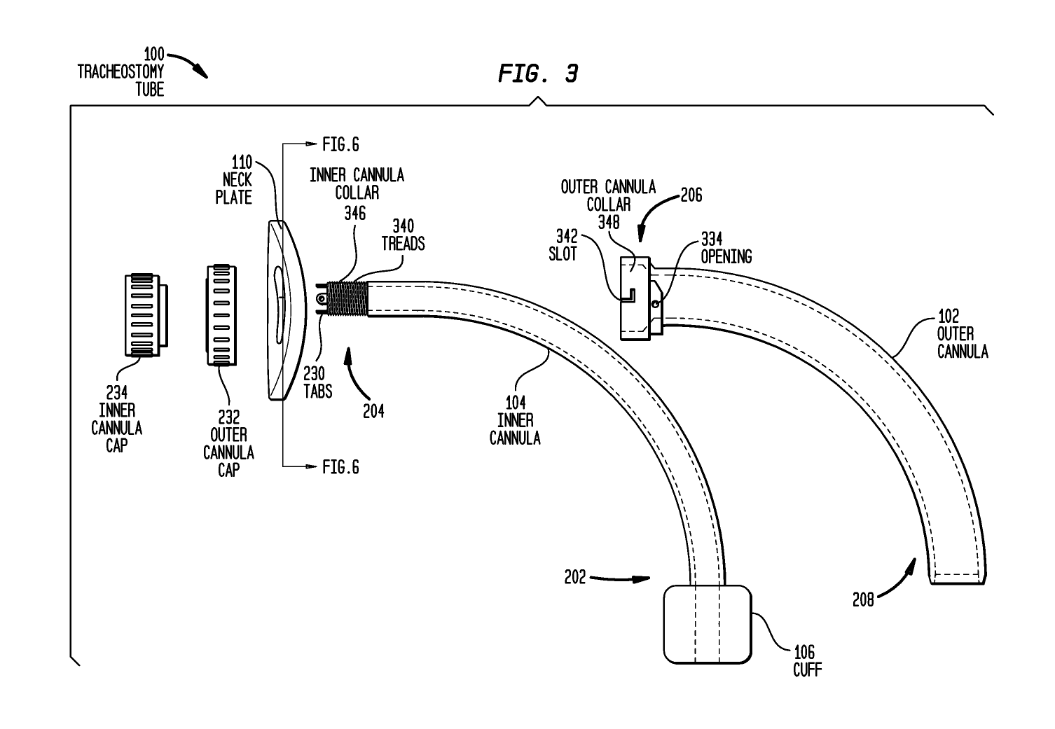

An exploded view of tracheostomy tube 100 is illustrated in FIG. 3. As noted, each connector 236, 238 comprises a cannula cap 234, 232 and a cannula collar. As shown in FIG. 3, outer cannula collar 348 is disposed on the proximal end 206 of outer cannula 102. Outer cannula collar 348 includes slots 342 therein which are used for coupling with outer cannula cap 232. An aperture 344 in proximal end 206 of outer cannula 102 is included to ensure that the air space within outer cannula 102 remains at atmospheric pressure when tracheostomy tube 100 is fully assembled and in place within patient 150. Proximal end 204 of inner cannula 104 includes inner cannula collar 346 which, in these embodiments, comprises threads 340 for coupling with inner cannula cap 234. Inner cannula 104 has a greater length than outer cannula 102, so that when inner cannula 104 is placed within outer cannula 102, distal end 202 of inner cannula 102 and cuff 106 extends beyond distal end 302 of outer cannula 102 and can be inflated to seal off trachea 152 as described above and in a manner that is well known to those of skill in the art.

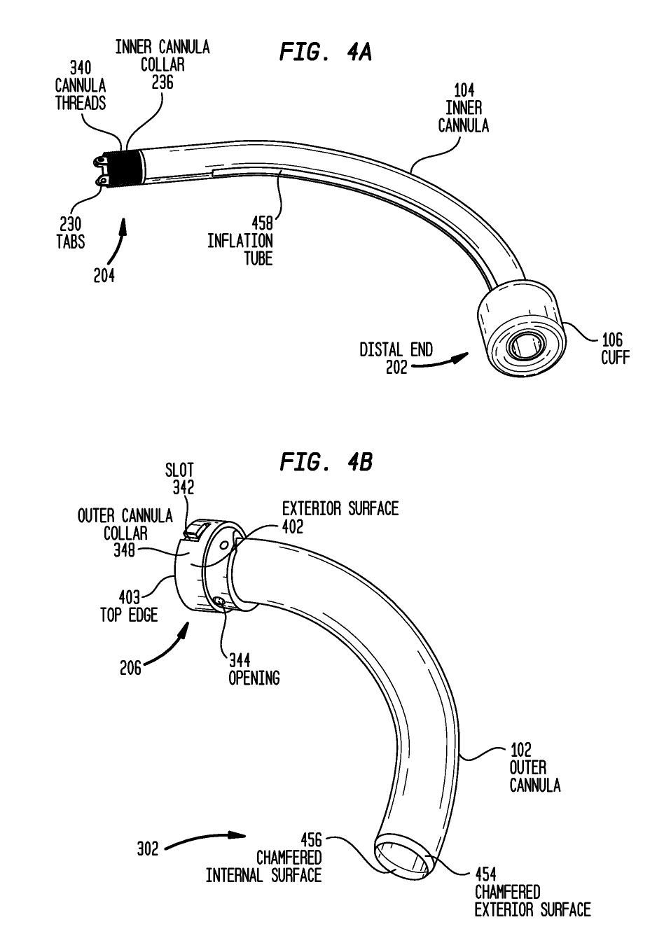

More detailed views of inner cannula 104 and outer cannula 102 are illustrated in FIGS. 4A and 4B, respectively. As shown in FIG. 4A, inner cannula 104 includes an inflation tube 458 running along its length. Inflation tube 458 enables cuff 106 to be fluidically connected to an air pump or other source of pressurized fluid (not shown) so that that cuff 106 can be inflated once tracheostomy tube 100 is inserted into patient 150. As seen in FIG. 4B, distal end 302 of outer cannula 102 has chamfered edges. The exterior surface 454 of distal end 302 is chamfered to facilitate insertion of outer cannula 102 into stoma 154 formed in patient 150. Internal surface 456 of distal end 302 is also chamfered to facilitate removal of inner cannula 104 from patient 150. Particularly, chamfered surface 456 aids in allowing cuff 106 to pass distal end 302 of outer cannula 102 as inner cannula 104 is withdrawn from outer cannula 102 when tracheostomy tube 100 is in place within patient 150.

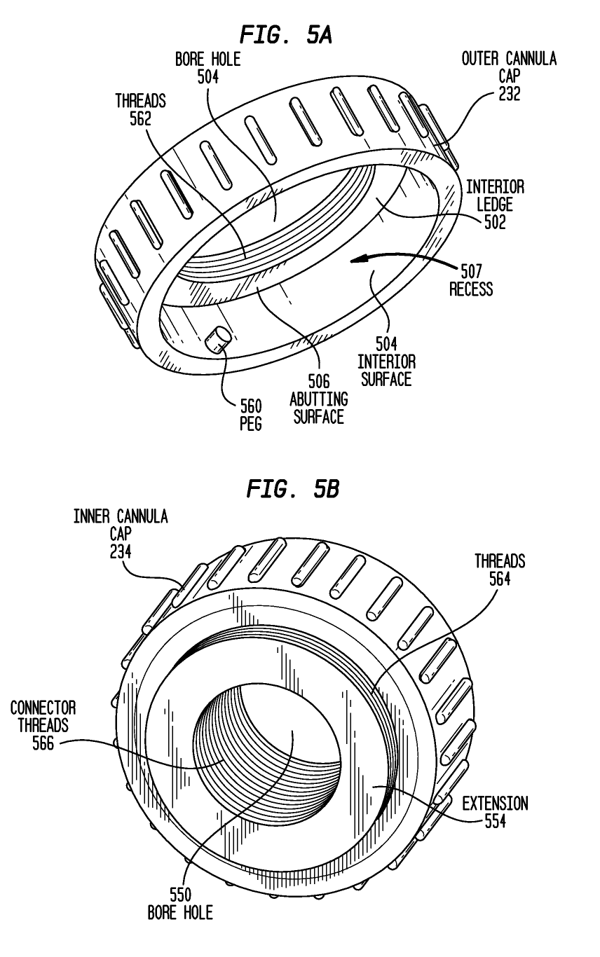

As noted above, interlocking mechanism 108 includes inner cannula connector 236 and outer cannula connector 238, each comprising a cannula cap 234, 232, respectively, and a cannula collar 346, 348, respectively. FIGS. 5A and 5B are perspective views of embodiments of outer cannula cap 232 and inner cannula cap 234 of interlocking mechanism 108. Outer cannula cap 232 includes one or more pegs 560 which are positioned to engage the complimentary slots 342 included at outer cannula collar 348 of outer cannula 102, as shown in FIG. 4B. Inward facing threads 562 are also included as part of outer cannula cap 232 so that it may be interlocked with complimentary threads 564 on inner cannula cap 234. Inner cannula cap 234 also includes a set of inward facing threads 566, which are complimentary to threads 340 on proximal end 204 of inner cannula 104 and permit inner cannula 104 to be coupled to inner cannula cap 234.

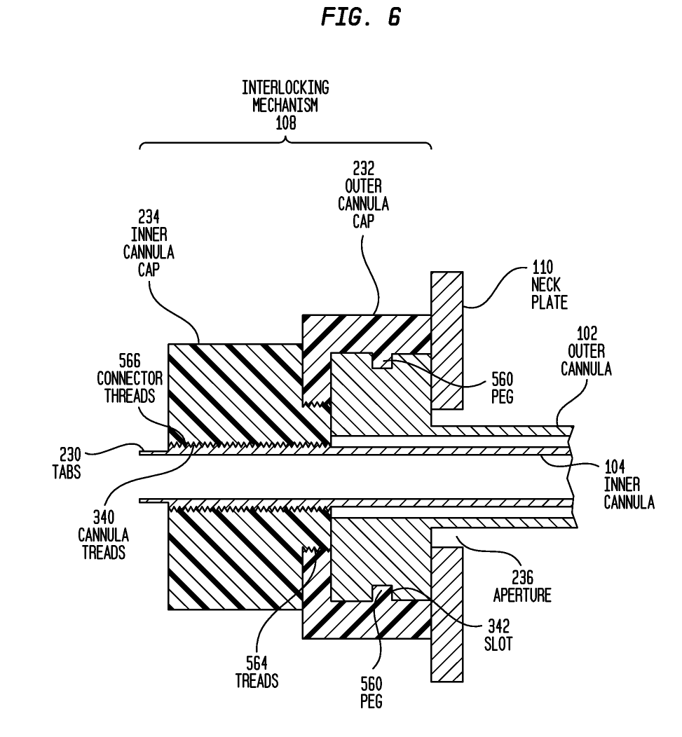

FIG. 6 is a cross-sectional view of interlocking mechanism 108 when in its “interlocked” configuration. In this figure, neck plate 110 is shown in cross-section, taken along section line 6-6 in FIG. 2, showing aperture 236 dimensioned to receive cannula 102 and cannula 104, and to provide a surface 602 against which components of interlocking mechanism 108 abut. As shown in FIG. 6, in their operative position, inner cannula 104 is located within outer cannula 102.

As noted, interlocking mechanism 108 comprises outer cannula cap 232 located at proximal end 206 of outer cannula 102, outer cannula collar 348 configured to releasably interlock with cap 232. Interlocking mechanism 108 further comprises inner cannula collar 236 comprising threads 340 located at the proximal end 204 of inner cannula 104. In the interlocked configuration, outer cannula cap 232 is secured to outer cannula collar 348, inner cannula cap 234 is secured to inner cannula collar 346, and the two caps 232, 234 are secured to each other. In this configuration, cannula 102 and 104 are prevented from moving relative to each other.

Pegs 560, as noted, radially extend inwardly from the interior surface 504 of outer cannula cap 232 to slidingly engage with slots 342 on exterior surface 402 of outer cannula collar 348. Outer cannula cap 232 is initially brought into contact with outer cannula 102 by positioning cap 232 on top of collar 348 such that top edge 403 of collar 348 is partially positioned within recess 507 until pegs 560 contact top edge 403 of collar 232. Manual rotation of cap 232 eventually brings pegs 560 into alignment with the opening of slots 342 at top edge 403 of collar 232. Collar 232 may then be advanced further into recess 507, causing pegs 560 to slide along the channel of slots 342 until pegs 560 contact the elbow of the slots. Additional rotation of cap 232 causes pegs 506 to travel along the laterally-extending channel of slots 342. Due to the interlocking of pegs 560 and slots 342, cap 232 cannot be removed from outer cannula 102 unless cap 232 is rotated in the opposite direction to bring pegs 560 back to the elbow of the slots. In that position pegs 560 are free to slide along the longitudinal channels of slots 342.

Similarly, inner cannula cap 234 has a threaded bore hole 550 and inner cannula 104 has threads 340 at its proximal end 204. Cap 234 is threaded onto cannula 104 until threads 340 are located at the proximal-most portion of threads 566.

To interlock inner cannula 104 to outer cannula 102, inner cannula cap 234 is secured to outer cannula cap 232. Outer cannula cap 232 has an interior ledge 502 extending radially inward. Ledge 502 has a bore hole 504 with threads 562 disposed on the inner surface of the bore hole. Interior ledge 502 has a bottom surface 506 that abuts against top edge 402 of collar 348. Threads 562 are disposed in the inner surface of ledge 502 as shown in FIG. 5A. Inner cannula cap 234 has a raised extension 554 with threads 564 disposed on the exterior surface to threadingly engage with threads 562 of outer cannula cap 232.

The above described coupling arrangement provides the proximal ends 206, 204, of outer cannula 102 and the inner cannula 104 with structural integrity to prevent relative movement between the two cannulae. Further, with this arrangement, neither of the outer cannula 102 nor inner cannula 104 can accidentally slide through aperture 236 in neck plate 110 to injure the patient while the two are coupled. During decoupling of cannulae 102, 104, threads 562 may be easily disengaged prior to disengagement of threads 566, such that the coupling between inner cannula cap 234 and inner cannula 104 may be maintained. This arrangement aids in reducing the accidental injury to the patient upon decoupling.

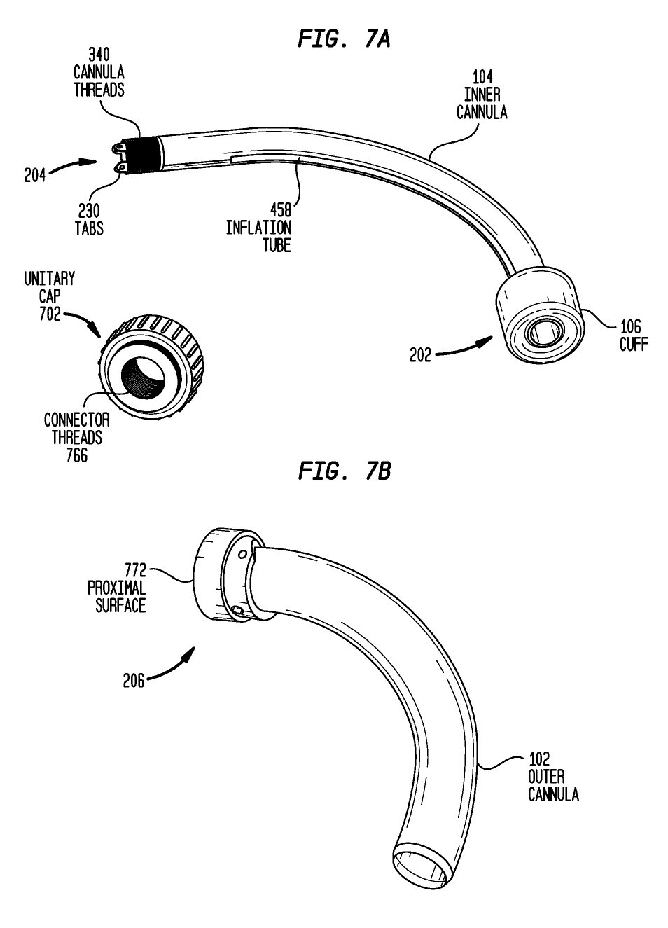

FIGS. 7A and 7B are perspective views of an alternative embodiment of interlocking mechanism 108, referred to herein as interlocking mechanism 708. Interlocking mechanism 708 comprises a unitary cap 702 configured to be removably secured to proximal end 204 of inner cannula 104. As shown in FIGS. 7A and 7B, outer cannula 102 has a proximal surface 772 which is sized to prevent outer cannula 102 from completely passing through aperture 236 in face plate 110. Cap 702 includes inward facing threads 766 which are complementary to the threads 340 at the proximal end of inner cannula 104. With this arrangement, cap 702, once coupled to proximal end 204 of inner cannula 104, abuts against proximal surface 772 of outer cannula 102. While this embodiment does not provide a direct coupling between inner cannula 104 and outer cannula 102, the two are loosely coupled once the cuff 106 is inflated and cap 702 abuts against proximal surface 772 of outer cannula 102.

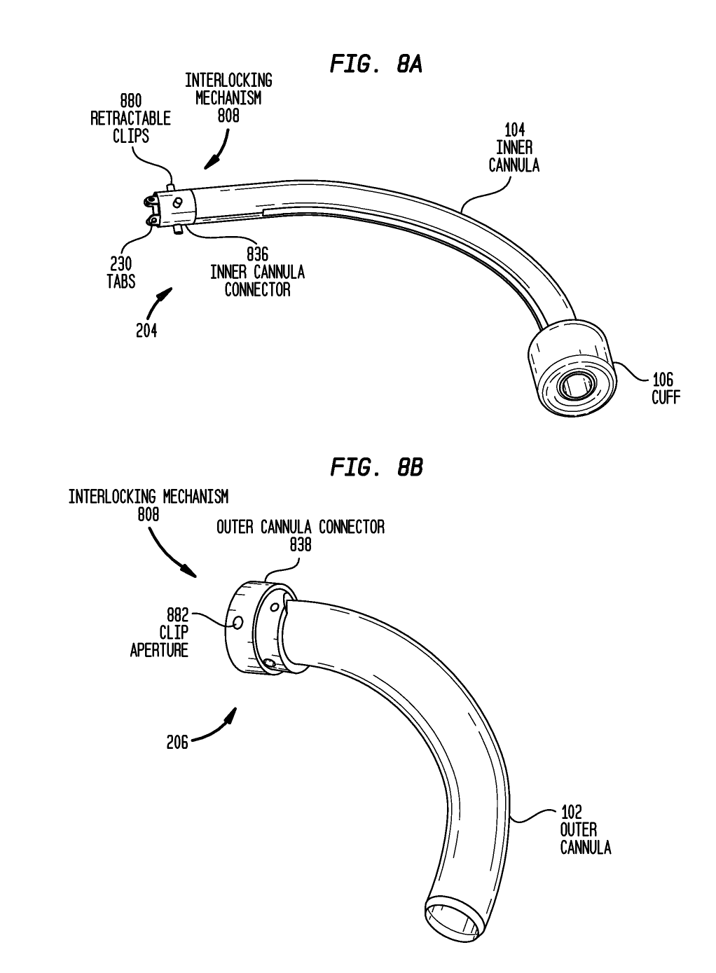

FIGS. 8A and 8B are perspective views of an alternative embodiment of interlocking mechanism 108, referred to herein as interlocking mechanism 808. Interlocking mechanism 808 comprises an inner cannula connector 836 and an outer cannula connector 838 disposed on the proximal ends 204, 206, respectively, of inner cannula 104 and outer cannula 102. In these embodiments, inner cannula connector 836 includes several retractable clips 880 which are arranged to engage clip apertures 882 of outer cannula connector 838. Retractable clips 880 are spring loaded so that they may be depressed for insertion into proximal end 206 of outer cannula 102 and subsequently extended outward again once clips 880 are aligned with apertures 882. To disconnect inner cannula 104 from outer cannula 102, clips 880 are depressed so that the clips disengage from apertures 882, thereby enabling extraction of inner cannula 104 from proximal end 206 of outer cannula 102.

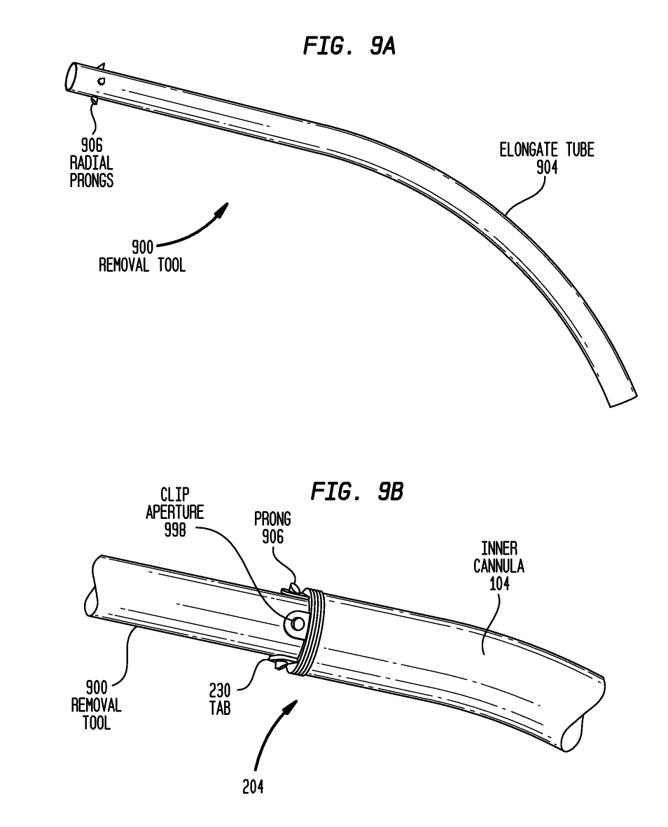

As noted above, in certain circumstances outer cannula 102 may be removed from a patient without removing inner cannula 104. FIGS. 9A and 9B illustrate an exemplary removal tool 900 which aids in removing outer cannula 102 from tracheostomy tube 100 while leaving inner cannula 104 in place. Removal tool 900, in its simplest form, is an elongate tube 904 that is approximately the same diameter as inner cannula 104. However, depending upon the method of coupling removal tool 900 to proximal end 204 of inner cannula 104, removal tool 900 may advantageously have a slightly larger or smaller outer diameter as compared to inner cannula 104.

As shown in FIG. 9A, removal tool 900 has a slightly smaller outer diameter than inner cannula 104 and includes radially extending prongs 906 at one end thereof, the prongs being positioned to engage the tabs 230 located at proximal end 204 of inner cannula 804. Prongs 906 engage tabs 230 by being inserted into apertures 998 included in each tab 230. When removal tool 900 is coupled to inner cannula 104, outer cannula 102 may be extracted from a patient while removal tool 990 is used to hold inner cannula 104 in position within the trachea of the patient, thereby reliably maintaining the stoma. During extraction, once removal tool 900 is coupled to inner cannula 104, outer cannula 102 is removed by being slid out of the patient's trachea and along the entire length of removal tool 900. As such, removal tool 900 passes entirely through outer cannula 102.

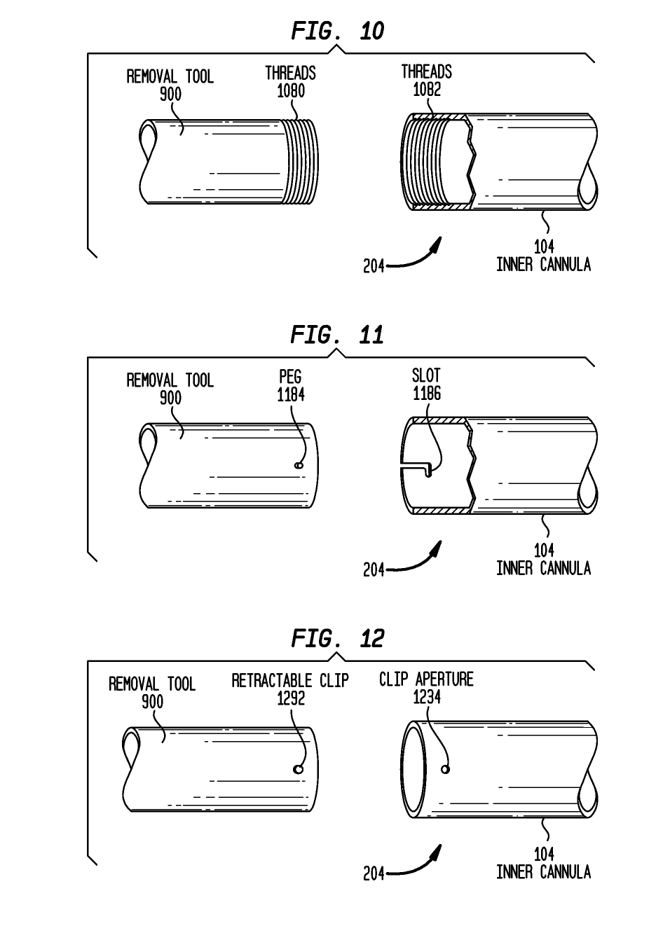

It should be appreciated that the coupling between removal tool 900 and the inner cannula 104 may be achieved through any one of a variety of different coupling arrangements. FIGS. 10-12 illustrate three different arrangements for coupling removal tool 900 to inner cannula 104.

In the illustrative embodiments of FIG. 10, removal tool 900 includes outer threads 1080 which are complementary to inner threads 1082 disposed in proximal end 204 of inner cannula 104. Removal tool is screwed into proximal end 204 so that threads 1080, 1082 mate with one another. Once coupled to one another, removal tool 900 holds inner cannula 104 in place during removal of outer cannula 102.

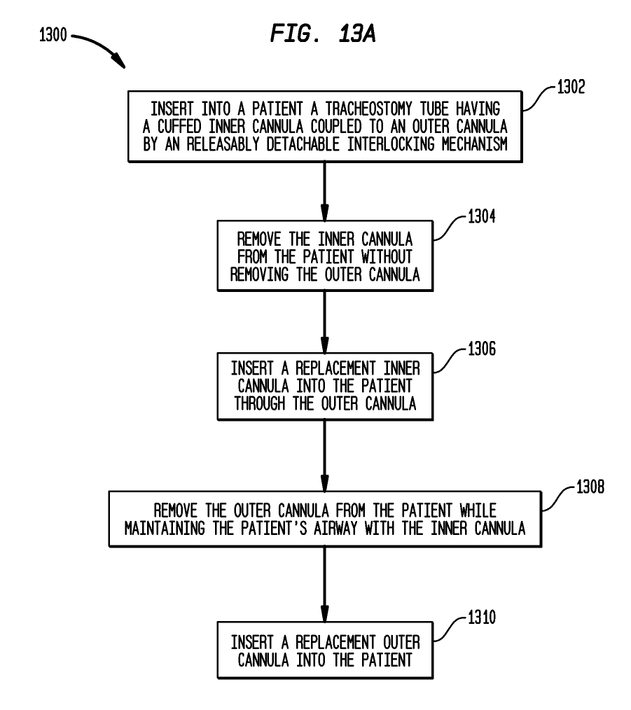

In the illustrative embodiments of FIG. 11, removal tool 900 includes pegs 1184 which are configured to engage slots 1186 in proximal end 204 of inner cannula 104. Once the pegs 1184 are engaged with the slots 1186, outer cannula 102 may be removed from the patient.

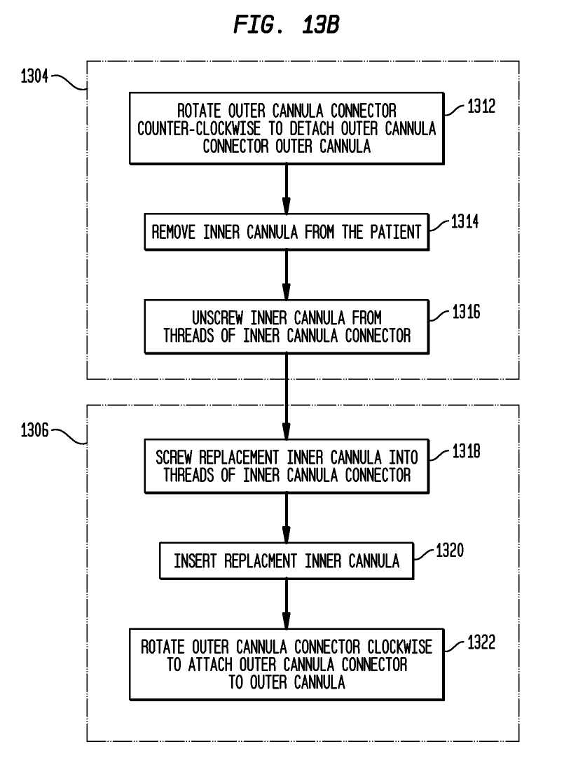

In the illustrative embodiments of FIG. 12, removal tool 900 includes retractable clips 1292 positioned to engage clip apertures 1234 in proximal end 204 of inner cannula 1204. These retractable clips 1292 and clip apertures 1234 function in the clips and corresponding apertures described with reference to FIGS. 8A and 8B. Specifically, clips 1292 are spring loaded so that they may be depressed for insertion into proximal end 204 of inner cannula 104 and subsequently extended outward again once clips 1292 are aligned with apertures 1234. To disconnect removal tool 900 from inner cannula 104, clips 1292 are depressed so that the clips disengage from apertures 1234.

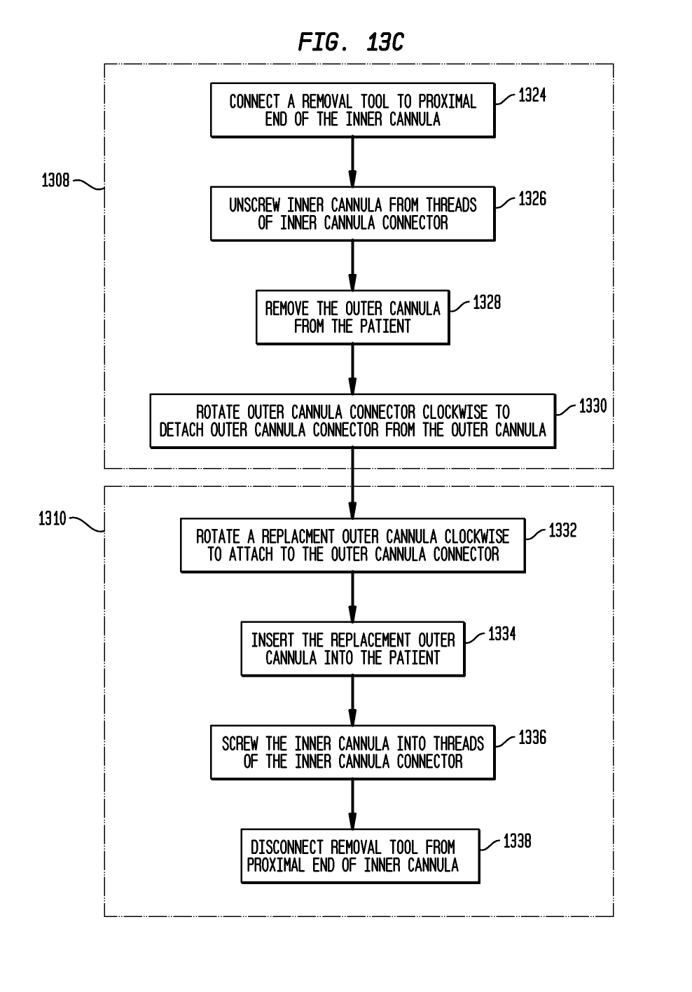

FIG. 13A is a flowchart showing the sequence of steps that may be implemented to utilize a tracheostomy tube of the present invention. At block 1302 the tracheostomy tube having a cuffed inner cannula and an outer cannula interlocked together by a releasably detachable interlocking mechanism. At block 1304, the inner cannula is removed from the patient without removing the outer cannula, and a replacement inner cannula is inserted into the outer cannula at block 1306. Following insertion of the replacement inner cannula, at block 1308 the outer cannula is removed from the patient while maintaining the patient's airway with the inner cannula. Then, at block 1310 a replacement outer cannula is inserted to the patient and secured to the replacement inner cannula.

FIG. 13B is a flowchart of the steps that may be performed at blocks 1304 and 1306 of FIG. 13A. In these illustrative embodiments, three steps are performed at each of blocks 1304 and 1306.

As noted with reference to FIG. 13A, at block 1304, the inner cannula is removed from the patient without removing the outer cannula. As shown in FIG. 13B, this involves rotating the outer cannula connector counter-clockwise to detach the outer cannula connector from the outer cannula. At block 1314 the inner cannula is removed from the outer cannula and the patient. Then, at block 1316 the inner cannula connector is unscrewed from the inner cannula.

As noted with reference to FIG. 13A, at block 1306, a replacement inner cannula is inserted in the patient. As shown in FIG. 13B, this includes screwing a replacement inner cannula into threads of the inner cannula connector at block 1318. Then, at block 1320, the replacement inner cannula is inserted into the patient. The outer cannula is then rotated clockwise to attach the outer cannula connector to the outer cannula.

FIG. 13C is a flowchart of the steps that may be performed at blocks 1308 and 1310 of FIG. 13A. In these illustrative embodiments, four steps are performed at each of blocks 1304 and 1306.

As noted with reference to FIG. 13A, at block 1308, the outer cannula is removed from the patient while maintaining the patient's airway with the inner cannula. As shown in FIG. 13C, this involves connecting a removal tool to the proximal end of the inner cannula at bloc 1324. At block 1326, the inner cannula is unscrewed from the threads of the inner cannula connector. Then, at block, the outer cannula is removed from the patient. Following removal, the outer cannula is rotated clockwise to detach outer cannula connector from the outer cannula.

As noted with reference to FIG. 13A, at block 1310, a replacement outer cannula is inserted to the patient and secured to the replacement inner cannula. As shown in FIG. 13C, this includes rotating a replacement outer cannula clockwise to attach to the outer cannula connector at block 1332. At block 1334, the replacement outer cannula is inserted into the patient. Then, at block 1336, the inner cannula is screwed into the threads of the inner cannula connector. The removal tool is then disconnected from the proximal end of the inner cannula at block 1338.

While various embodiments of the present invention have been described above, it should be understood that they have been presented by way of example only, and not limitation. It will be apparent to persons skilled in the relevant art that various changes in form and detail may be made therein without departing from the spirit and scope of the invention. Thus, the breadth and scope of the present invention should not be limited by any of the above-described exemplary embodiments, but should be defined only in accordance with the following claims and their equivalents. All patents and publications discussed herein are incorporated in their entirety by reference thereto.

What is claimed is:

1. A tracheostomy tube for use with a neck plate having an aperture, comprising:
an elongate outer cannula having a lumen and configured to extend through the aperture; and
an elongate inner cannula having a lumen and an inflatable cuff, and configured to extend through the lumen of the outer cannula such that the cuff extends beyond a distal end of the outer cannula.

2. The tracheostomy tube of claim 1, further comprising:
an interlocking mechanism configured to releasably secure a proximal end of the inner cannula to a proximal end of the outer cannula.

3. The tracheostomy tube of claim 2, wherein the interlocking mechanism comprises:
a first connector releasably coupled to a proximal end of the outer cannula; and
a second connector releasably coupled to a proximal end of the inner cannula,
wherein the first and second connectors releasably interlock with each other.

4. The tracheostomy tube of claim 3, wherein the first and second connectors are threadably coupled to one another.

5. The tracheostomy tube of claim 3, wherein the first connector and the proximal end of the outer cannula are coupled using a peg and slot arrangement.

6. The tracheostomy tube of claim 3, wherein the second connector and the proximal end of the inner cannula are threadably coupled.

7. The tracheostomy tube of claim 1, wherein a proximal end of the outer cannula is configured to not be passable through the aperture.

8. The tracheostomy tube of claim 1, wherein each of the cannulae are independently removable from the aperture when the cannulae are in place within a trachea.

9. A tracheostomy tube comprising:
a neck plate including an aperture;
an outer cannula extending through the aperture;
a first connector releasably coupled to a proximal end of the outer cannula;
an inner cannula extending through the outer cannula and the aperture;
a second connector releasably coupling a proximal end of the inner cannula to the outer cannula, wherein each of the cannulae are independently removable from the aperture when the cannulae are in place within a trachea; and
an inflatable cuff affixed to a distal end of the inner cannula.

10. The tracheostomy tube of claim 9, wherein the proximal end of the outer cannula is configured to not be passable through the aperture.

11. The tracheostomy tube of claim 9, wherein the first and second connectors interlock.

12. The tracheostomy tube of claim 9, wherein the first and second connectors are threadably coupled to one another.

13. The tracheostomy tube of claim 9, further comprising a removal tool adapted to couple to the proximal end of the inner cannula.

14. The tracheostomy tube of claim 9, wherein the first connector and the outer cannula are coupled using a peg and slot arrangement.

15. The tracheostomy tube of claim 9, wherein the second connector and the inner cannula are threadably coupled.

16. The tube of claim 9, wherein a distal end of the outer cannula is chamfered on at least one of an inner rim and an outer rim.

17. The tube of claim 9, wherein the inner cannula comprises PVC.

18. The tube of claim 9, wherein the outer cannula comprises flexible silicon.

19. A kit comprising,
a tracheostomy tube for use with a neck plate having an aperture, comprising:
an elongate outer cannula having a lumen and configured to extend through the aperture;
an elongate inner cannula having a lumen and an inflatable cuff, and configured to extend through the lumen of the outer cannula such that the cuff extends beyond a distal end of the outer cannula; and
an interlocking mechanism configured to releasably secure a proximal end of the inner cannula to a proximal end of the outer cannula, and
a removal tool configured to be coupled to the proximal end of the inner cannula.

20. The kit of claim 19, wherein the interlocking mechanism comprises:
a first connector releasably coupled to a proximal end of the outer cannula; and
a second connector releasably coupled to a proximal end of the inner cannula,
wherein the first and second connectors releasably interlock with each other.

21. The kit of claim 20, wherein the first and second connectors are threadably coupled to one another.

22. The kit of claim 20, wherein the first connector and the proximal end of the outer cannula are coupled using a peg and slot arrangement.

23. The kit of claim 20, wherein the second connector and the proximal end of the inner cannula are threadably coupled.

24. The kit of claim 19, wherein a proximal end of the outer cannula is configured to not be passable through the aperture.

25. The kit of claim 19, wherein each of the cannulae are independently removable from the aperture when the cannulae are in place within a trachea.

26. The kit of claim 19, wherein the removal tool is adapted to hold the inner cannula in place within the aperture while the outer cannula is removed from the aperture.

27. The kit of claim 19, wherein the removal tool is configured to be passable through the outer cannula.

28. The kit of claim 19, wherein the removal tool comprises a tube.

29. The kit of claim 19, wherein the inner cannula and the removal tool are adapted to be coupled using a peg and aperture arrangement.

30. The kit of claim 19, wherein the inner cannula and the removal tool are adapted to be coupled using a peg and slot arrangement.

31. The kit of claim 19, wherein the inner cannula and the removal tool are adapted to be coupled using a retractable clip and aperture arrangement.

32. The kit of claim 19, wherein the inner cannula and the removal tool are adapted to be threadably coupled.
